(19) United States
(12) Patent Application Publication
(10) Pub. No.: US 2020/0337729 A1
(43) Pub. Date: Oct. 29, 2020

(54) SURGICAL INSTRUMENT FOR TRANSCERVICAL EVALUATION OF UTERINE MOBILITY

(71) Applicant: Covidien LP, Mansfield, MA (US)

(72) Inventors: Nikolai D. Begg, Wellesley, MA (US); Rebecca D. White, Kennett Square, PA (US); Lisa M. Quealy, Dracut, MA (US); Jacob J. Kelly, North Easton, MA (US); Jordan A. Whisler, Brookline, MA (US); Chad A. Pickering, Woburn, MA (US)

(21) Appl. No.: 16/857,297

(22) Filed: Apr. 24, 2020

Related U.S. Application Data
(60) Provisional application No. 62/839,711, filed on Apr. 28, 2019.

(51) Int. Cl. A61B 17/42

(52) U.S. Cl. CPC A61B 17/4241; A61B 2090/066

(57) ABSTRACT

A uterine manipulator includes a body defining proximal and distal end portions, a handle operably coupled to the proximal end portion, an end effector assembly operably coupled and configured to articulate relative to the distal end portion, and a force sensor configured to measure a force indicative of a torque required to articulate the end effector assembly relative to the distal end portion of the body. A method of transcervically determining uterine mobility includes transcervically positioning a surgical instrument such that an end effector assembly of the surgical instrument extends into a uterus, articulating the end effector assembly relative to a body of the surgical instrument to thereby move the uterus, and measuring a force indicative of a torque required to move the uterus.

### CROSS-REFERENCE TO RELATED APPLICATIONS

This application claims the benefit of, and priority to, U.S. Provisional Patent Application No. 62/839,711 filed on Apr. 28, 2019, the entire contents of which is hereby incorporated by reference.

### BACKGROUND

### Technical Field

The present disclosure relates to surgical instruments. More specifically, the present disclosure relates to a surgical instrument such as, for example, a uterine manipulator, configured to facilitate transcervical evaluation of uterine mobility.

### Background of Related Art

Various surgical instruments are utilized to perform transcervical diagnostic and/or therapeutic surgical tasks. Uterine manipulators, for example, are often utilized in laparoscopic hysterectomy procedures for, among other tasks, positioning the uterus such that a colpotomy can be performed and the uterus removed. Uterine manipulators typically include a handle, a body extending distally from the handle, and an end effector assembly that is configured for insertion through the cervix and into the uterus. Some uterine manipulators are configured to permit articulation of the end effector relative to the body.

### SUMMARY

As used herein, the term “distal” refers to the portion that is being described which is further from a user, while the term “proximal” refers to the portion that is being described which is closer to a user. Further, to the extent consistent, any of the aspects described herein may be used in conjunction with any or all of the other aspects described herein.

Provided in accordance with aspects of the present disclosure is a uterine manipulator including a body defining a proximal end portion and a distal end portion, a handle operably coupled to the proximal end portion of the body, an end effector assembly operably coupled to the distal end portion of the body and configured to articulate relative to the distal end portion of the body, and a force sensor configured to measure a force indicative of a torque required to articulate the end effector assembly relative to the distal end portion of the body.

In an aspect of the present disclosure, the end effector assembly is pivotable relative to the body about a distal pivot. In such aspects, the force sensor may be configured to measure a torque at the distal pivot as the force indicative of the torque required to articulate the end effector assembly relative to the distal end portion of the body.

In another aspect of the present disclosure, the handle is pivotable relative to the proximal end portion of the body about a proximal pivot to thereby articulate the end effector assembly relative to the distal end portion of the body. In such aspects, the force sensor may be configured to measure a torque at the proximal pivot as the force indicative of the torque required to articulate the end effector assembly relative to the distal end portion of the body.

In yet another aspect of the present disclosure, the end effector assembly includes an elongated shaft extending to an atraumatic distal tip and an inflatable balloon disposed on the elongated shaft and proximally-spaced from the atraumatic distal tip.

In still another aspect of the present disclosure, the uterine manipulator further includes a distal connector pivotably coupled to the distal end portion of the body. In such aspects, the end effector assembly may extend distally from the distal connector such that pivoting of the distal connector relative to the distal end portion of the body articulates the end effector assembly relative to the distal end portion of the body. Further, in aspects, a stop may be disposed about the distal connector.

In still yet another aspect of the present disclosure, the force sensor is a torque transducer. The torque transducer may include at least one strain gauge.

In another aspect of the present disclosure, a console is communicatively coupled to the force sensor and configured to receive the measured force from the force sensor. The console may be configured to determine the torque required to articulate the end effector assembly based upon the received measured force and display, store, and/or output the determined torque.

Also provided in accordance with aspects of the present disclosure is a method of transcervically determining uterine mobility. The method includes transcervically positioning a surgical instrument such that an end effector assembly of the surgical instrument extends into a uterus, articulating the end effector assembly relative to a body of the surgical instrument to thereby move the uterus, and measuring a force indicative of a torque required to move the uterus.

In an aspect of the present disclosure, the method further includes stabilizing the end effector assembly within the uterus after transcervically positioning the surgical instrument and before articulating the end effector assembly.

In another aspect of the present disclosure, the method further includes determining the torque required to move the uterus based upon the indicate force measured.

In still another aspect of the present disclosure the method further includes displaying, storing, and/or outputting the determined torque. The determined torque, in aspects, is displayed, stored, and/or output as a numerical value, chart, or graph.

In yet another aspect of the present disclosure, articulating the end effector assembly includes articulating the end effector assembly through a range of motion and measuring the force includes measuring a plurality of forces throughout articulation of the end effector assembly throughout the range of motion.

In still yet another aspect of the present disclosure, the method further includes determine torque information based upon the measured plurality of forces.

### BRIEF DESCRIPTION OF THE DRAWINGS

Various aspects and features of the present disclosure are described hereinbelow with reference to the drawings wherein like numerals designate identical or corresponding elements in each of the several views.

FIG. 1 is a side view of a uterine manipulator provided in accordance with the present disclosure; and

FIG. 2 is a transverse, cross-sectional view of a distal portion of the uterine manipulator of FIG. 1 shown extending transcervically into a uterus and disposed in an articulated position, thereby manipulating the uterus.

### DETAILED DESCRIPTION

The present disclosure provides a surgical instrument, e.g., a uterine manipulator, configured to facilitate transcervical evaluation of uterine mobility. Although the aspects and features of the present disclosure are detailed below with respect to a uterine manipulator, it is contemplated that the aspects and features of the present disclosure may likewise be utilized with any other suitable surgical instrument. Further, although the uterine manipulator of the present disclosure is detailed hereinbelow for the purpose of facilitating transcervical evaluation of uterine mobility, the uterine manipulator of the present disclosure is also configured to facilitate performance of other surgical tasks typically accomplished at least in part using a uterine manipulator, e.g., biopsy or other diagnostic procedures; tubal ligations; treatment of endometriosis; removal of adhesions, fibroids, or cysts; hysterectomies; etc.

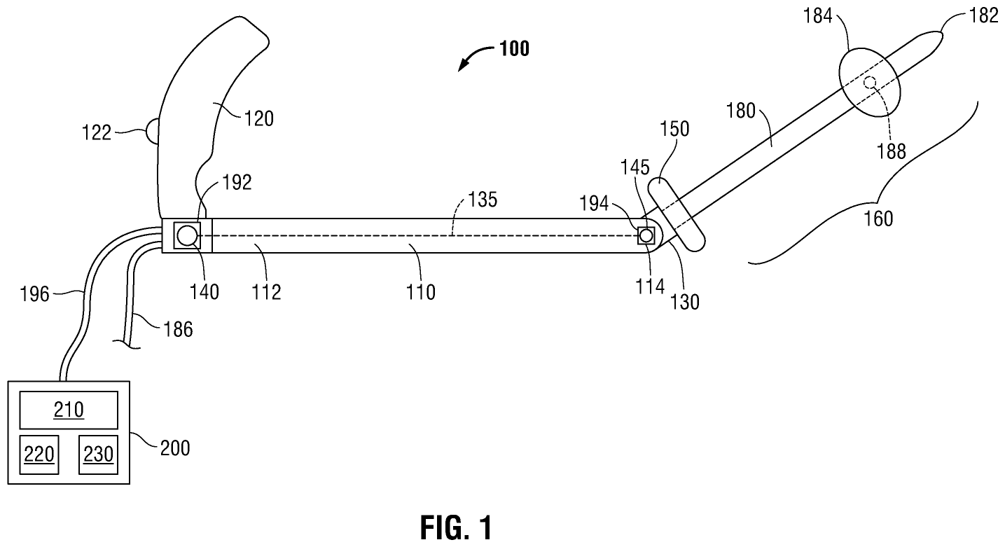

Turning to FIG. 1, a uterine manipulator provided in accordance with the present disclosure is shown generally identified by reference numeral 100. Uterine manipulator 100 includes a body 110, a handle 120 disposed at a proximal end portion 112 of body 110, a distal connector 130 disposed at a distal end portion 114 of body 110, and an end effector assembly 160 extending distally from distal connector 130. Uterine manipulator 100 further includes a proximal pivot 140 pivotably coupling handle 120 with body 110 at proximal end portion 112 of body 110 and a distal pivot 145 pivotably coupling distal connector 130 with body 110 at distal end portion 114 of body 110. An actuation linkage 135, e.g., a cable, rod, or other suitable link, extends through body 110 and operably couples handle 120 with distal connector 130 to enable pivoting of distal connector 130 relative to body 110 in response to pivoting of handle 120 relative to body 110. More specifically, pivoting of handle 120 relative to body 110 about proximal pivot 140 correspondingly pivots distal connector 130 relative to body 110 about distal pivot 145. Other suitable configurations for pivoting distal connector 130 relative to body 110 in response to manipulation of handle 120 (or a portion thereof) are also contemplated such as, for example, other mechanical configurations, motor-driven configurations, etc. Further, gearing or other suitable mechanisms may be provided to amplify or attenuate the pivoting of distal connector 130 in response to manipulation of handle 120. Handle 120 may be pivotable in either direction from an at-rest position relative to body 110, e.g., to thereby enable pivoting of distal connector 130 in either direction relative to body 110 from its at-rest position. Alternatively, handle 120 may be configured to pivot in a single direction from its at-rest position relative to body 110, e.g., to thereby limit pivoting of distal connector 130 in a single direction relative to body 110 from its at-rest position.

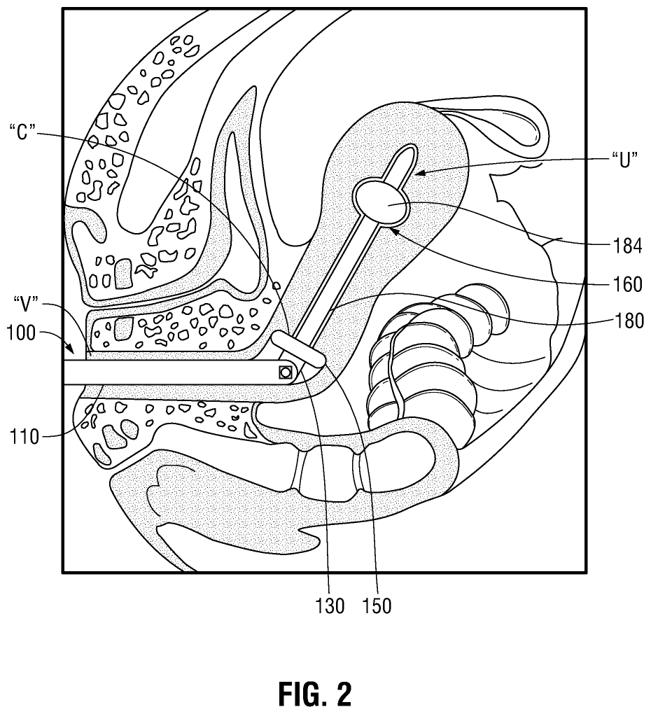

Uterine manipulator 100 further includes a stop 150 supported on distal connector 130 although, in embodiments, stop 150 may alternatively be disposed on body 110 towards distal end portion 114 thereof. In use, stop 150 is configured to abut the cervix “C” (FIG. 2), e.g., to stabilize the uterus “U” (FIG. 2), to define a maximum insertion depth of uterine manipulator 100, and/or to prevent the loss of insufflation gases from the uterus “U” (FIG. 2). Stop 150 may be configured as an occluder (a foam occluder, an inflatable occluder, etc.), a colpotomy cup, or may define any other suitable configuration.

Uterine manipulator 100 additionally includes an end effector assembly 160 that is engaged with and extends distally from distal connector 130. In embodiments, end effector assembly 160 is releasably engagable with distal connector 130. End effector assembly 160 includes an elongated shaft 180 defining an atraumatic distal tip 182 configured to facilitate insertion of end effector assembly 160 through the cervix “C” (FIG. 2) and into the uterus “U” (FIG. 2). A balloon 184 is supported on elongated shaft 180 towards but proximally-spaced from atraumatic distal tip 182. Balloon 184 is adapted to connect to a fluid source (not shown) by way of a tube 186 and an aperture 188. More specifically, tube 185 connects to the fluid source (not shown) and extends through body 110 and end effector assembly 160 to aperture 188. Aperture 188 communicates with tube 186 and an interior of balloon 184 to enable selective inflation or deflation of balloon 184. Balloon 184 may be inflated, for example, to expand into contact with the interior wall of the uterus “U” to stabilize end effector assembly 160 therein such that manipulation of end effector assembly 160, e.g., via pivoting handle 120 relative to body 110, likewise manipulates the uterus “U.” An activation button 122 disposed on handle 120 enables the selective control, e.g., ON/OFF, inflate/deflate, etc., of balloon 184.

Continuing with reference to FIG. 1, a force sensor 192, 194, e.g., a torque sensor, is operably associated with proximal pivot 140 and/or distal pivot 145, respectively, to enable measurement of the force, e.g., torque, required to pivot distal connector 130 about distal pivot 145 and relative to body 110 (directly at distal pivot 145 via force sensor 194 and/or indirectly at proximal pivot 140 via force sensor 192) and, thus, to enable determination of the force, e.g., torque, required to articulate end effector assembly 160 relative to body 110. Force sensors 192, 194 (or additional sensors) may further be configured to measure the degree of pivoting about proximal pivot 140 and/or distal pivot 145, respectively, to enable correlation of the measured force, e.g., torque, required to articulate end effector assembly 160 relative to body 110 with the articulated position of end effector assembly 160 relative to body 110.

Force sensors 192, 194 may be configured as torque transducers configured to convert the mechanical torque of proximal and distal pivots 140, 145, respectively, (e.g., the inputs) into electrical signals (e.g., the outputs) using, for example, one or more strain gauges. The output electrical signals, indicative of the corresponding mechanical torque inputs, are communicated to a console 200 via a wired connection, e.g., using electrical cable 196, or via a wireless connection. Console 200 may be a stand-alone hardware unit (as shown), may be incorporated into uterine manipulator 100, e.g., on or within handle 120, or may be a virtual machine. Regardless of its particular configuration, console 200 is configured to receive the output electrical signals from sensors 192, 194 and, based thereon, determine torque information based thereupon, and/or provide a suitable output indicating the determined torque information. To this end, console 200 includes a display 210, a controller 220, and a memory 230.

Display 210 is configured to display the output of console 200 indicating the torque information and may be disposed on console 200 (as shown), disposed on uterine manipulator 100, e.g., handle 120 thereof, or may be an external display device connected (via wired or wireless connection) to console 200. Display 210, more specifically, may be configured to provide: a numerical display of the torque information as one or more torque values, e.g., a current torque measurement, previous torque measurement(s), a high torque measurement, and/or a low torque measurement; a table displaying the torque information as torque value(s) versus articulated position(s) of end effector assembly 160 (e.g., at increments throughout the angular range of motion of end effector assembly 160); a graphical display indicating torque information as torque values versus articulated positions of end effector assembly 160 (e.g., continuously or incrementally throughout the angular range of motion of end effector assembly 160); or combinations thereof. Other suitable display outputs are also contemplated. Additionally or alternatively, memory 230 may store the torque information, e.g., the one or more torque values, tables, graphs, etc., for subsequent retrieval, display, printing, communication, or other output.

Controller 220 of console 200 includes an input/output, a processor, and a storage device associated with the processor. The input/output is configured to receive the electrical signals output from sensors 192, 194 and to provide outputs to display 210, memory 230, and/or other device(s). The storage device stores one or more programs for execution by the processor to perform the various functions of console 200 such as, for example, determining the torque information based upon the output electrical signals from sensors 192, 194.

As an alternative to the above-detailed processor-based console 200, other suitable consoles 200 may be provided such as, for example, a console configured as a potentiometer configured to receive the electrical signals from sensors 192, 194 and output a response indicative of a torque information (in absolute or relative terms), e.g., as a visual response, audible response, or other suitable response. As another example, sensors 192, 194 may be configured as mechanical sensors with console 200 configured as a mechanical dial configured to display the torque information (in absolute or relative terms).

With additional reference to FIG. 2, in use, uterine manipulator 100, lead by end effector assembly 160, is inserted through the vagina “V,” cervix “C,” and into the uterus “U” such that stop 150 abuts or is disposed in proximity to the cervix “C” while effector assembly 160 extends therethrough into the uterus “U.” In this position, body 110 extends through the vagina “V,” while handle 120 (FIG. 1) remains externally disposed to permit manipulation thereof by the user. Thereafter, balloon 184 may be inflated to expand into contact with the interior wall of the uterus “U,” thus stabilizing end effector assembly 160 relative to the uterus “U.”

With uterine manipulator 100 positioned as detailed above, handle 120 (FIG. 1) is manipulated to thereby pivot distal connector 130 relative to body 110 such that end effector assembly 160 is articulated through at least a portion of its angular range of motion. As end effector assembly 160 is articulated through its angular range of motion, the uterus “U” is likewise moved due to the stabilization of end effector assembly 160 within the uterus “U.” However, the uterus “U” exerts a resistive force to this movement. Thus, in order to articulate end effector assembly 160 and move the uterus “U,” sufficient torque must be applied at distal pivot 145 (and a correspondingly sufficient torque at proximal pivot 140; see FIG. 1) in order to overcome the resistive force of the uterus “U” and enable articulation of end effector assembly 160 and movement of the uterus “U.”

Force sensor 192 and/or force sensor 194 (FIG. 1), as detailed above, are configured to measure the force at proximal and/or distal pivots 140, 145 (FIG. 1), respectively, as end effector assembly 160 is articulated through at least a portion of its angular range of motion to thereby move the uterus “U” through a corresponding range of motion. This measured force data is communicated to console 200 for determination of the corresponding torque information based thereupon and for display, storage, and/or output of the determined torque information.

The determined torque information is indicative of uterine mobility in that the torque required to move the uterus “U” through at least a portion of its range of motion is dependent upon the resistive force of the uterus “U.” The resistive force of the uterus “U,” in turn, may be dependent upon conditions associated with the uterus “U.” For example, pelvic adhesions due to endometriosis may limit the mobility of uterus “U” and, thus, result in greater resistive forces (compared to a baseline) as reflected by the determined torque information. The determined torque information may not only indicate the presence of adhesions but may also indicate the severity of the adhesions. As another example, laxity of support ligaments may result in lower resistive forces (compared to a baseline) as reflected by the determined torque information, and may be indicative of pelvic organ prolapse risk and/or severity. However, the present disclosure is not limited to the above examples; rather, it is understood that the determined torque information may be utilized to facilitate the evaluation and/or diagnosis of trauma, diseases, conditions, etc., that impact uterine mobility.

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure. Additionally, the elements and features shown or described in connection with certain embodiments may be combined with the elements and features of certain other embodiments without departing from the scope of the present disclosure, and that such modifications and variations are also included within the scope of the present disclosure. Accordingly, the subject matter of the present disclosure is not limited by what has been particularly shown and described.

What is claimed is:

1. A uterine manipulator, comprising:
a body defining a proximal end portion and a distal end portion;
a handle operably coupled to the proximal end portion of the body;
an end effector assembly operably coupled to the distal end portion of the body and configured to articulate relative to the distal end portion of the body; and
a force sensor configured to measure a force indicative of a torque required to articulate the end effector assembly relative to the distal end portion of the body.

2. The uterine manipulator according to claim 1, wherein the end effector assembly is pivotable relative to the body about a distal pivot, and wherein the force sensor is configured to measure a torque at the distal pivot as the force indicative of the torque required to articulate the end effector assembly relative to the distal end portion of the body.

3. The uterine manipulator according to claim 1, wherein the handle is pivotable relative to the proximal end portion of the body about a proximal pivot to thereby articulate the end effector assembly relative to the distal end portion of the body, and wherein the force sensor is configured to measure a torque at the proximal pivot as the force indicative of the torque required to articulate the end effector assembly relative to the distal end portion of the body.

4. The uterine manipulator according to claim 1, wherein the end effector assembly includes:
an elongated shaft extending to an atraumatic distal tip; and
an inflatable balloon disposed on the elongated shaft and proximally-spaced from the atraumatic distal tip.

5. The uterine manipulator according to claim 1, further including a distal connector pivotably coupled to the distal end portion of the body, wherein the end effector assembly extends distally from the distal connector such that pivoting of the distal connector relative to the distal end portion of the body articulates the end effector assembly relative to the distal end portion of the body.

6. The uterine manipulator according to claim 5, further including a stop disposed about the distal connector.

7. The uterine manipulator according to claim 1, wherein the force sensor is a torque transducer.

8. The uterine manipulator according to claim 7, wherein the torque transducer includes at least one strain gauge.

9. The uterine manipulator according to claim 1, further including a console communicatively coupled to the force sensor and configured to receive the measured force from the force sensor.

10. The uterine manipulator according to claim 9, wherein the console is configured to determine the torque required to articulate the end effector assembly based upon the received measured force.

11. The uterine manipulator according to claim 10, wherein the console is configured to at least one of: display, store, or output the determined torque.

12. A method of transcervically determining uterine mobility, comprising:
transcervically positioning a surgical instrument such that an end effector assembly of the surgical instrument extends into a uterus;
articulating the end effector assembly relative to a body of the surgical instrument to thereby move the uterus; and
measuring a force indicative of a torque required to move the uterus.

13. The method according to claim 12, further including stabilizing the end effector assembly within the uterus after transcervically positioning the surgical instrument and before articulating the end effector assembly.

14. The method according to claim 12, further including determining the torque required to move the uterus based upon the indicate force measured.

15. The method according to claim 14, further including at least one of: displaying, storing, or outputting the determined torque.

16. The method according to claim 15, wherein the determined torque is at least one of displayed, stored, or output as a numerical value, chart, or graph.

17. The method according to claim 12, wherein:
articulating the end effector assembly includes articulating the end effector assembly through a range of motion; and
measuring the force includes measuring a plurality of forces throughout articulation of the end effector assembly throughout the range of motion.

18. The method according to claim 17, further including determine torque information based upon the measured plurality of forces.
